(19) United States
(12) Patent Application Publication
(10) Pub. No.: US 2014/0255453 A1
(43) Pub. Date: Sep. 11, 2014

(54) HERBAL OINTMENT FOR MUSCULOSKELETAL AND JOINT-RELATED CONDITIONS

(71) Applicant: JAXSEN'S LLC, ADAMSTOWN, MD (US)

(72) Inventors: MICHAEL J. URSCHEL, ADAMSTOWN, MD (US); TARA L. URSCHEL, ADAMSTOWN, MD (US); KRAIG D. MOORE, UPPER MALBORO, MD (US)

(73) Assignee: JAXSEN'S LLC, ADAMSTOWN, MD (US)

(21) Appl. No.: 13/794,149

(22) Filed: Mar. 11, 2013

(51) Int. Cl. A61K 36/355; A61K 45/06; A61K 36/28; A61K 36/30; A61K 36/185

(52) U.S. Cl. CPC A61K 36/355; A61K 36/30; A61K 36/185; A61K 36/28; A61K 45/06
USPC 424/400; 424/725; 424/757; 424/730; 424/764; 604/290

(57) ABSTRACT

An herbal ointment. The herbal ointment may include about 40-45 weight percent of herb-infused oil, water or alcohol, or any desired combination thereof, about 30 weight percent purified water, about 10 weight percent emulsifier wax, about 5-10 weight percent menthol, about 3 weight percent dimethyl isosorbide, about 2 weight percent glycerin, about 2 weight percent hydrogenated methyl abietate, about 0.5 weight percent lonicera caprifolium and lonicera japanica extract, about 0.5 weight percent tocopherol, about 0.35 weight percent vanillyl butyl ether, about 0.35 weight percent xanthan gum, about 0.3 weight percent citric acid 50% aqueous solution, and about 0.25 weight percent menthyl lactate, the herb-infused oil, water or alcohol including a 1:5 dilution of herbal extracts in a solvent, wherein the herbal extracts are of German Chamomile (Matricaria recutita), Valerian (Valeriana officinalis), Ginger (Zingiber officinale Roscoe), Peppermint (Mentha.times.piperita), Feverfew (Tanacetum parthenium), and Lemon Balm (Melissa officinalis). The ointment may further include Glucosamine hydrochloride, chondroitin sulfate sodium and methyl sulfone methane.

### CROSS-REFERENCE TO RELATED APPLICATIONS

This application is a continuation-in-part of U.S. patent application Ser. No. 13/034,728, filed Feb. 25, 2011 and entitled HERBAL OINTMENT FOR MUSCULOSKELETAL AND JOINT-RELATED CONDITIONS, which claims priority to U.S. Provisional Application 61/308,424, filed Feb. 26, 2010 and entitled APPARATUS AND METHODS FOR NON-INVASIVE PHYSICAL TREATMENT, and U.S. Provisional Application 61/314,837, filed Mar. 17, 2010 and entitled APPARATUS AND METHODS FOR NON-INVASIVE PHYSICAL TREATMENT AND OINTMENTS THEREFORE, the entire contents of which are hereby incorporated by reference.

### BACKGROUND

Aging individuals generally begin to suffer from increased aches and pains arising out of the musculoskeletal system. Furthermore, injuries resulting from accidents, falls, sports and the like can impart damage to the musculoskeletal system that may manifest themselves not only as pain but also as stress, tension, general discomfort, and other maladies. Surgical procedures designed to remedy such symptoms and the causes thereof are generally expensive and invasive, causing further stress and discomfort to the patient, while not necessarily achieving the desired results. Therefore, an effective, non-invasive, and natural solution for relieving symptoms related to the musculoskeletal system is needed.

### SUMMARY

According to at least one exemplary embodiment, an herbal ointment for musculoskeletal and joint-related conditions is provided. The ointment may include at least one of an herb-infused oil, water or alcohol having extracts from a plurality of herbs therein. The herbs may include, but are not limited to Comfrey (Symphytum officinale), Calendula Flower (Calendula officinalis), German Chamomile (Matricaria recutita), Stinging Nettle Leaf (Urtica dioica), Arnica montana, Devil's Claw (Harpagophytum procumbens), and Tumeric (Curcuma longa).

According to another exemplary embodiment, the herbs may include, but are not limited to, German Chamomile (Matricaria recutita), Valerian (Valeriana officinalis), Ginger (Zingiber officinale Roscoe), Peppermint (Mentha.times.piperita), Feverfew (Tanacetum parthenium), and Lemon Balm (Melissa officinalis). Additional herbs included in the herbal ointment may include Licorice (Glycyrrhiza glabra), Boswellia (Boswellia serrata), White Willow Bark (Salix alba), Witch Hazel (Hamamelis virginiana), St. John's Wart (Hypericum perforatum), Black Cohosh (Actaea racemosa), Hops (Humulus lupulus), Saffron (Crocus sativas) Passion Flower (Passiflora incarnata), and Rosemary (Rosmarinus officinalis). In some embodiments, the ointment may include, but is not limited to, glucosamine hydrochloride and chondroitin sulfate sodium (“glucosamine-chondroitin”) and methyl sulfone methane (“MSM”), dimethyl sulfone (“DMSO”) or the like.

### DETAILED DESCRIPTION

Aspects of the invention are disclosed in the following description and related drawings directed to specific embodiments of the invention. Alternate embodiments may be devised without departing from the spirit or the scope of the invention. Additionally, well-known elements of exemplary embodiments of the invention will not be described in detail or will be omitted so as not to obscure the relevant details of the invention. Further, to facilitate an understanding of the description discussion of several terms used herein follows.

As used herein, the word “exemplary” means “serving as an example, instance or illustration.” The embodiments described herein are not limiting, but rather are exemplary only. It should be understood that the described embodiment are not necessarily to be construed as preferred or advantageous over other embodiments. Moreover, the terms “embodiments of the invention”, “embodiments” or “invention” do not require that all embodiments of the invention include the discussed feature, advantage or mode of operation.

According to at least one exemplary embodiment, an herbal ointment is disclosed. The herbal ointment is adapted to be used for treatment and amelioration of symptoms related to musculoskeletal and joint-related conditions and injuries. Such conditions and injuries may include, but are not limited to: osteoarthritis; rheumatoid arthritis; gouty arthritis; muscle strains, sprains, and spasms; ligament sprains, strains or tears; as well as aches, pains and general discomfort related to the musculoskeletal system.

Additionally, the herbal ointment may be used by an individual to treat, for example, sports related injuries, degenerative joint diseases, and chronic muscle spasms as well as to speed the healing of injuries, maintain a desired level of health and to increase an individual's athletic performance. The herbal ointments may also be used for anti-inflammatory purposes.

A first exemplary embodiment of the herbal ointment may include a plurality of herbal extract ingredients directed towards treatment and amelioration of symptoms related to, for example, musculoskeletal and joint-related conditions and injuries. The herbs from which the ingredients may be extracted may include, but are not limited to, Comfrey (Symphytum officinale), Calendula Flower (Calendula officinalis), German chamomile (Matricaria recutita), Stinging Nettle Leaf (Urtica dioica), Wolfs bane (Arnica montana), Devil's Claw (Harpagophytum procumbens), Tumeric (Curcuma longa) and Rosemary (Rosmarinus officinalis).

The above-listed herbal active ingredients may be selected for their particular medicinal and therapeutic properties. Comfrey (Symphytum officinale) is an Allantoin containing anti-inflammatory herb that can speed healing of soft torn tissue, such as, but not limited to ligaments, tendons and muscles, and that can influence the course of bone ailments. Additionally, the allantoin contained in Comfrey (Symphytum officinale) may speed up the natural replacement of body cells. Calendula Flower (Calendula officinalis), has anti-inflammatory properties and can further control bleeding and soothe irritated tissue. German Chamomile (Matricaria recutita) is an inhibitor of the COX-2 enzyme, and has anti-inflammatory properties as well as spasmolytic, and anxiolytic effects, and can therefore induce muscle relaxation and relief of skeletal muscle spasms and associated pain. Stinging Nettle Leaf (Urtica dioica) can inhibit the breakdown of articular joint cartilage via the inhibition of matrix metalloproteinases as well as the inhibition of the NF-kappa B protein complex, and is therefore beneficial in arthritis treatment and rheumatism pain relief. Devil's Claw (Harpagophytum procumbens) has anti-inflammatory properties and can further preserve articular cartilage via down-regulation of Tumor Necrosis Factor Alpha and other interleukins. Devil's Claw (Harpagophytum procumbens) can thus reduce pain and aid in the treatment of arthritis as well as chronic low back pain. Tumeric (Curcuma longa) has anti-inflammatory, antiseptic and analgesic properties and can further have benefits for treatment of burns, cuts, and bruises as well as arthritic conditions. Arnica montana is an anti-inflammatory herb that can speed healing of soft torn tissue, such as ligaments, tendons and muscles. Rosemary (Rosmarinus officinalis) is an anti-inflammatory and a pain relieving herb. The above-listed herbal ingredients may be used to prepare an herbal-infused oil, water or alcohol, or any other desired combination thereof, prior to combination with any other desired active ingredients and any desired inactive ingredients.

A second exemplary embodiment of the herbal ointment may include a plurality of herbal extract ingredients directed towards relieving muscle pains and spasms. The herbs from which the ingredients may be extracted may include, but are not limited to, German chamomile (Matricaria recutita), Valerian (Valeriana officinalis), Ginger (Zingiber officinale Roscoe), Peppermint (Mentha.times.piperita), Feverfew (Tanacetum parthenium), and Lemon balm (Melissa officinalis).

The above-listed herbal active ingredients may be selected for their particular medicinal and therapeutic properties. German Chamomile (Matricaria recutita) is an inhibitor of the COX-2 enzyme, and has anti-inflammatory properties as well as spasmolytic, and anxiolytic effects, and can therefore induce muscle relaxation and relief of skeletal muscle spasms. Valerian (Valeriana officinalis) has anti-spasmodic properties, and is also a sedative and anticonvulsant. Ginger (Zingiber officinale Roscoe) has analgesic effects and can also be used to reduce inflammation and reduce pains from arthritis. Peppermint (Mentha.times.piperita) has soothing and anti-inflammatory properties on the skin surface and is capable of easily penetrating the skin and tissues to provide anti-spasmodic and relaxant effects on the muscles. Feverfew (Tanacetum parthenium) likewise has analgesic and anti-spasmodic properties and is beneficial for treatment of arthritis. Lemon balm (Melissa officinalis) has analgesic properties as well as anxiolytic and relaxant effects.

Additional active ingredients may be included in the embodiments of the herbal ointment. Such ingredients may include, but are not limited to: menthol, dimethyl isosorbide, hydrogenated methyl abietate, vanillyl butyl ether, menthyl lactate, glucosamine hydrochloride, chondroitin sulfate sodium, methyl sulfone methane, dimethyl suflone, or the like. These ingredients have been selected to further enhance the efficacy of the herbal ointment. Menthol is a topical analgesic having local anesthetic and counter-irritant qualities. Vanillyl butyl ether is a warming agent, while menthyl lactate is a cooling agent, and the combination of the two ingredients can be used to provide simultaneous heating sensations and cooling sensations to the area where the herbal ointment is applied. Glucosamine hydrochloride and chondroitin sulfate sodium (“Glucosamine-chondroitin”), and methyl sulfone methane (“MSM”) or dimethyl sulfone (“DMSO”) assist in preserving joints and treating pain and debilitation associated with osteoarthritis. In some embodiments, the glucosamine hydrochloride may be about 0.2 weight percent, chondroitin sulfate sodium may be about 0.2 weight percent, and the methyl sulfone methane or dimethyl sulfone may be about 0.2 weight percent. In some embodiments Dimethyl isosorbide is a penetrating agent that can facilitate penetration of the skin by the herbal ointment, thereby enhancing the delivery of the active ingredients of the herbal ointment to the desired areas. Hydrogenated methyl abietate is a delayed release agent and film former, and functions as a fixative, carrier and stabilizer for the herbal ointment.

In addition to the ingredients described above, embodiments of the herbal ointment may also include, if desired, at least one of the following ingredients: camphor, beeswax, titanium dioxide, pokeroot (phytolacca), cleavers (galium aparine), licorice (glycyrrhiza glabra), autumn saffron crocus (crocus sativus), boswellia, bromelain enzymes, white willow bark (salix alba), witch-hazel (Hamamelis virginiana), capsaicin, St. John's Wort (hypericum perforatum), black cohosh (actaea racemosa), hops (humulus lupulus), passionflower (passiflora), carotenoids, and bioflavonoids.

The preparation of the herbal ointment may involve a plurality of steps. The herbs of the above-described embodiments, as well as any other desired herbs, may be used to prepare an herb-infused oil, an herb-infused water or an herb-infused alcohol, or any desired combination thereof, such as, but not limited to an herb-infused oil and water combination. The herb-infused oil, water or alcohol, or combination thereof, may further be combined with the additional active ingredients, as well as any other desired active or inactive ingredients, so as to form the herbal ointment.

To prepare the herb-infused oil, water or alcohol, or any desired combination thereof, the herbs of the above-described embodiments may be placed into a solvent in a ratio of approximately 1 part herbs to 5 parts solvent so as to form a 1:5 dilution. The solvent may be, but is not limited to, fractionated coconut oil, distilled water, an alcohol, or glycerol. Regarding the infused oil preparation, the resultant mixture may be heated to a temperature of between about 100 degrees Fahrenheit (about 37.78° C.) to about 115 degrees Fahrenheit (about 46.11° C.). The mixture may be maintained at this temperature for approximately two weeks and may be stirred for approximately 15 minutes every hour during this time. The extraction of the herbal ingredients from their respective herbs and into the solvent may thus be optimized. Next, the resultant infusion may be pressed through a press, for example a tincture press, so as to obtain a pressed infusion. The pressed infused oil, water or alcohol, or combination thereof, may then be stored in a dark, cool and dry place for approximately 24-48 hours. Subsequently, the pressed infused oil, water or alcohol, or combination thereof, may be strained through a porous medium, for example a cheese cloth, gauze, mesh sieve or the like. The resultant herb-infused oil, water or alcohol, or combination thereof, may then be stored in a dark, cool and dry place, or combined with the additional active ingredients as well as any other desired active or inactive ingredients, so as to form the herbal ointment. Alternatively, the herb extraction may be prepared by Spagyric methods, or any other desired solvent extraction processes.

To prepare the herbal ointment, the combination of herbal infused oil, water or alcohol, or combinations thereof, with the additional active ingredients as well as any other desired active or inactive ingredients, may be performed by formation of three separate phases, wherein certain desired ingredients are combined so as to form a first phase, other certain desired ingredients are combined separately from the first phase so as to form a second phase, and yet other certain desired ingredients are combined separately from the first and second phases so as to form a third phase. Upon formation of the three separate phases, the phases may be treated and combined according to the below-described procedure.

Desired ingredients for the first phase and their corresponding approximate proportions by weight relative to the resultant weight of the herbal ointment, may be as follows:

| Ingredient | % w/w (approx.) |
|---|---|
| Purified water | 30.75 |
| Citric acid 50% aqueous solution | 0.3 |
| Honeysuckle extract | 0.5 |
| (Lonicera caprifolium and | |
| Lonicera japanica) | |
| Xanthan gum | 0.35 |
| Menthol | 5.0 |
| Glycerin 99.7% | 2.0 |

Desired ingredients for the second phase and their corresponding approximate proportions by weight relative to the resultant weight of the herbal ointment, may be as follows:

| Ingredient | % w/w (approx.) |
|---|---|
| Herb-infused oil/water/alcohol | 45.0 |
| Dimethyl isosorbide | 3.0 |
| Tocopherol (Vitamin E) | 0.5 |
| Hydrogenated methyl abietate | 2.0 |
| Emusifier wax | 10.0 |

Desired ingredients for the third phase and their corresponding approximate proportions by weight relative to the resultant weight of the herbal ointment, may be as follows:

| Ingredient | % w/w (approx.) |
|---|---|
| Vanillyl butyl ether | 0.35 |
| Menthyl lactate | 0.25 |

The ingredients for the first phase may be combined in a first vessel. The proportion of citric acid may be adjusted such that the pH of the solution is approximately 5.5.+−.0.5. The solution may then be heated to approximately 80° C. until the solution is uniform. The ingredients of the second phase may be combined in a second vessel and heated to approximately 75° C.-78° C. until the solution is substantially uniform. The solution of the second phase may then be added to the solution of the first phase and stirred for approximately 5 minutes, for example by a propeller or vane disposed in the vessel, as well as by side sweep mixing. Subsequently, the combined first and second phase solution may be stirred continuously for about 5-10 minutes, for example by a homogenizer at about 5,000 rpm, or at any other desired speed. The mixing may also be facilitated by a propeller or vane disposed in the vessel, as well as by side sweep mixing. Subsequently, the solution may be cooled to approximately 40° C.-45° C., at which point the ingredients of the third phase may be added, with continuous stirring of the solution. The resultant herbal ointment may then be cooled to approximately 30° C. and stirring may continue until the herbal ointment is substantially uniform.

The above described-embodiments of the herbal ointment may be applied directly to the skin of a subject, proximate to the affected area, so as to provide relief for the symptoms related to the conditions described herein. However, other modes of application may also be used as desired. For example, the embodiments of the herbal ointment may be used in conjunction with an applicator, such as a roll-on stick, spray or any other desired applicator. The embodiments of the herbal ointment may also be used in conjunction with a patch, such as a transdermal patch. The transdermal patch (single layer drug-in-adhesive or multi-layer drug-in-adhesive systems) may include a reservoir layer that contains the herbal ointment, an adhesive portion for adhering to the skin of the subject around the affected area, a backing portion for handling the transdermal patch, and an occlusive layer that may facilitate increasing the humidity around the application site, thereby facilitating the increase of transdermal absorption of the herbal ointment. The embodiments of the herbal ointment may also be impregnated into an electrode pad, which may be any type of electrode pad known in the art and may be a type of electrode pad that does not require a conductive gel for operation.

In some embodiments the herbal ointment includes the combination of Comfrey, Devil's Claw, Fevrefew and Urtica diocia with dimethyl isosorbide. Preferably, in some embodiments, one or more additional herbs are included in a combination with the foregoing. Preferably, in some embodiments, hydrogenated methyl abietate is included in a combination with the foregoing. Preferably, in some embodiments, menthyl lactate and vanilyl butyl ether are further included in a combination with the foregoing.

Exemplary percentage weights of the various ingredients have been described. Other percentage weights are possible within the scope of the invention. All of the ingredients are not required to be present in any given embodiment of the disclosed herbal ointment.

In some embodiments, the weight percentage of the herb-infused oil/water/alcohol may be about 42.5 percent. In some embodiments, it is at least 30 weight percent. In some embodiments, it may be between about 40 and 45 weight percent. In some embodiments, it may be less than about 75 weight percent. In some embodiments, the weight percentage may be within a range between any of the foregoing values.

In some embodiments, weight percentage of Menthol is about 5 percent. In some, it is at least 5 weight percent. In some, it is less than about 15 weight percent. In some embodiments, it is about 7.5 weight percent. In some embodiments, the weight percentage may be within a range between any of the foregoing values. In some embodiments, menthol is not present, but peppermint is. In some embodiments, increased percentage of Menthol is accompanied by the presence of vanillyl butyl ether, or an increased percentage of vanillyl butyl ether.

In some embodiments, weight percentage of purified water is about 30%. In some embodiments, it is at least about 10 weight percent. In some embodiments, it is less than about 95 weight percent. In some embodiments, it may be less than about 70 weight percent. In some embodiments, the weight percentage may be within a range between any of the foregoing values.

In some embodiments, weight percentage of dimethyl isosorbide is about 3 percent. In some embodiments it is at least 0.1 weight percent. In some embodiments, it is less than 10 weight percent. In some, it is less than about 5 weight percent. In some embodiments, the weight percentage may be within a range between any of the foregoing values.

In some embodiments, weight percentage of glycerin is 2 percent. In some embodiments, it is at least 0.1 weight percent. In some, it is less than about 10 weight percent. In some embodiments it is less than about 3 weight percent. In some embodiments, the weight percentage may be within a range between any of the foregoing values.

In some embodiments, weight percentage of hydrogenated methyl abietate is about 2 percent. In some embodiments, it is at least 0.1 weight percent. In some, it is less than about 5 weight percent. In some embodiments it is at least about 1 weight percent. In some embodiments, the weight percentage may be within a range between any of the foregoing values.

In some embodiments, weight percentage of lonicera caprifolium and lonicera japanica extract is about 0.5 percent. In some embodiments, it is at least about 0.1 weight percent. In some embodiments, it is less than about 1 weight percent. In some embodiments, it is at least about 0.35 weight percent. In some embodiments, the weight percentage may be within a range between any of the foregoing values.

In some embodiments, weight percentage of tocopherol is about 0.5 percent. In some, it is at least 0.01 weight percent. In some, it is less than about 3 weight percent. In some embodiments, it is at least 0.25 weight percent. In some embodiments, the weight percentage may be within a range between any of the foregoing values.

In some embodiments, weight percentage of vanillyl butyl ether is about 0.35 weight percent. In some, it is at least 0.01 weight percent. In some, it is less than about 3 weight percent. In some embodiments, it is at least about 0.2 weight percent. In some embodiments, the weight percentage may be within a range between any of the foregoing values.

In some embodiment, weight percentage of xanthum gum is about 0.35 percent. In some, it is at least 0.01 weight percent. In some, it is less than about 1 weight percent. In some it is at least about 0.2 weight percent. In some embodiments, the weight percentage may be within a range between any of the foregoing values.

In some embodiments, the weight percentage of citric acid in a 50% aqueous solution is about 0.3 percent. In some embodiments, it is at least 0.01 weight percent. In some, it is less than about 2 weight percent. In some embodiments it is at least about 0.1 weight percent. In some embodiments, the weight percentage may be within a range between any of the foregoing values.

In some embodiments, the weight percentage of menthyl lactate is about 0.25 percent. In some embodiments, it is at least 0.01 weight percent. In some, it is less than about 3 weight percent. In some embodiments it is at least about 0.1 weight percent. In some embodiments, the weight percentage may be within a range between any of the foregoing values.

In some embodiments, the weight percentage of emulsifying wax is about 10 percent. In some, it is at least 0.5 weight percent. In some embodiments, it is less than about 20 weight percent. In some embodiments it is at least about 5 weight percent. In some embodiments, the weight percentage may be within a range between any of the foregoing values.

In some embodiments, the glucosamine hydrochloride may be about 0.2 weight percent, chondroitin sulfate sodium may be about 0.2 weight percent, and the methyl sulfone methane or dimethyl sulfone may be about 0.2 weight percent. In some embodiments, the weight percentage of one or more of the foregoing is at least 0.01 weight percent. In some embodiments, the weight percentage of one or more of the foregoing is less than approximately 0.5 weight percent. In some embodiments, the weight percentage may be within a range between any of the foregoing values.

The foregoing description and accompanying figures illustrate the principles, preferred embodiments and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

We claim:

1. An herbal ointment for relief of symptoms related to musculoskeletal and joint-related conditions, comprising:
about 45 weight percent of at least one of an herb-infused oil, water and alcohol; about 30 weight percent purified water;
about 10 weight percent emulsifier wax;
about 5 weight percent menthol;
about 3 weight percent dimethyl isosorbide;
about 2 weight percent glycerin;
about 2 weight percent hydrogenated methyl abietate;
about 0.5 weight percent lonicera caprifolium and lonicera japanica extract;
about 0.5 weight percent tocopherol; about 0.35 weight percent vanillyl butyl ether;
about 0.35 weight percent xanthan gum;
about 0.3 weight percent citric acid 50% aqueous solution; and
about 0.25 weight percent menthyl lactate;
the at least one of the herb-infused oil, water and alcohol comprising a 1:5 dilution of herbal extracts in a solvent, wherein the herbal extracts are of Comfrey (Symphytum officinale), Calendula Flower (Calendula officinalis), German Chamomile (Matricaria recutita), Stinging Nettle Leaf (Urtica dioica), Arnica montana, Devil's Claw (Harpagophytum procumbens), and Tumeric (Curcuma longa).

2. The herbal ointment of claim 1, wherein the solvent is selected from the group of fractionated coconut oil, distilled water, an alcohol, or glycerol.

3. The herbal ointment of claim 1, wherein the at least one of the herb-infused oil, water and alcohol further comprises extracts of at least one of Licorice (Glycyrrhiza glabra), Boswellia (Boswellia serrata), White Willow Bark (Salix alba), Witch Hazel (Hamamelis virginiana), St. John's Wart (Hypericum perforatum), Black Cohosh (Actaea racemosa), Hops (Humulus lupulus), Saffron (Crocus sativas) and Passion Flower (Passiflora incarnata)

4. An herbal ointment for relief of symptoms related to musculoskeletal and joint-related conditions, comprising:
about 45 weight percent of at least one of an herb-infused oil, water and alcohol;
about 30 weight percent purified water; about 10 weight percent emulsifier wax; about 5 weight percent menthol;
about 3 weight percent dimethyl isosorbide;
about 2 weight percent glycerin;
about 2 weight percent hydrogenated methyl abietate;
about 0.5 weight percent lonicera caprifolium and lonicera japanica extract;
about 0.5 weight percent tocopherol;
about 0.35 weight percent vanillyl butyl ether; about 0.35 weight percent xanthan gum;
about 0.3 weight percent citric acid 50% aqueous solution; and
about 0.25 weight percent menthyl lactate;
the at least one of the herb-infused oil, water and alcohol comprising a 1:5 dilution of herbal extracts in a solvent, wherein the herbal extracts are of German Chamomile (Matricaria recutita), Valerian (Valeriana officinalis), Ginger (Zingiber officinale Roscoe), Peppermint (Mentha.times.piperita), Feverfew (Tanacetum parthenium), and Lemon Balm (Melissa officinalis).

5. The method of claim 4, wherein the solvent is selected from the group of fractionated coconut oil, distilled water, an alcohol, or glycerol.

6. The herbal ointment of claim 4, wherein the at least one of the herb-infused oil, water and alcohol further comprises extracts of at least one of phytolacca, galium aparine, glycyrrhiza glabra, crocus sativus, boswellia, salix alba, Hamamelis virginiana, hypericum perforatum, actaea racemosa, humulus lupulus, and passiflora.

7. A method of providing relief of symptoms related to musculoskeletal and joint-related conditions, comprising:
providing an herbal ointment comprising about 45 weight percent of at least one of an herb-infused oil, water and alcohol, about 30 weight percent purified water, about 10 weight percent emulsifier wax, about 5 weight percent menthol, about 3 weight percent dimethyl isosorbide, about 2 weight percent glycerin, about 2 weight percent hydrogenated methyl abietate, about 0.5 weight percent lonicera caprifolium and lonicera japanica extract, about 0.5 weight percent tocopherol, about 0.35 weight percent vanillyl butyl ether, about 0.35 weight percent xanthan gum, about 0.3 weight percent citric acid 50% aqueous solution, and about 0.25 weight percent menthyl lactate, the at least one of the herb-infused oil, water and alcohol comprising a 1:5 dilution of herbal extracts in a solvent, wherein the herbal extracts are extracts of Comfrey (Symphytum officinale), Calendula Flower (Calendula officinalis), German Chamomile (Matricaria recutita), Stinging Nettle Leaf (Urtica dioica), Arnica montana, Devil's Claw (Harpagophytum procumbens), and Tumeric (Curcuma longa),
applying an effective amount of the herbal ointment to the affected area on a person's body.

8. The method of claim 7, wherein the solvent is selected from the group of fractionated coconut oil, distilled water, an alcohol, or glycerol.

9. The method of claim 7, wherein the at least one of the herb-infused oil, water and alcohol further comprises extracts of at least one of phytolacca, galium aparine, glycyrrhiza glabra, crocus sativus, boswellia, salix alba, Hamamelis virginiana, hypericum perforatum, actaea racemosa, humulus lupulus, and passiflora.

10. The method of claim 7, wherein the herbal ointment is disposed within a transdermal patch.

11. The method of claim 7, wherein the herbal ointment is disposed within at least one of a roll-on stick and a spray apparatus.

12. The method of claim 7, wherein the herbal ointment is disposed within an electrode pad.

13. A method of providing relief of symptoms related to musculoskeletal and joint-related conditions, comprising:
providing an herbal ointment comprising about 45 weight percent of at least one of an herb-infused oil, water and alcohol, about 30 weight percent purified water, about 10 weight percent emulsifier wax, about 5 weight percent menthol, about 3 weight percent dimethyl isosorbide, about 2 weight percent glycerin, about 2 weight percent hydrogenated methyl abietate, about 0.5 weight percent lonicera caprifolium and lonicera japanica extract, about 0.5 weight percent tocopherol, about 0.35 weight percent vanillyl butyl ether, about 0.35 weight percent xanthan gum, about 0.3 weight percent citric acid 50% aqueous solution, and about 0.25 weight percent menthyl lactate, the at least one of the herb-infused oil, water and alcohol comprising a 1:5 dilution of herbal extracts in a solvent, wherein the herbal extracts are of German Chamomile (Matricaria recutita), Valerian (Valeriana officinalis), Ginger (Zingiber officinale Roscoe), Peppermint (Mentha.times.piperita), Feverfew (Tanacetum parthenium), and Lemon Balm (Melissa officinalis),
applying an effective amount of the herbal ointment to the affected area on a person's body.

14. The method of claim 13, wherein the solvent is selected from the group of fractionated coconut oil, distilled water, an alcohol, or glycerol.

15. The method of claim 13, wherein the at least one of the herb-infused oil, water and alcohol further comprises extracts of at least one of phytolacca, galium aparine, glycyrrhiza glabra, crocus sativus, boswellia, salix alba, Hamamelis virginiana, hypericum perforatum, actaea racemosa, humulus lupulus, and passiflora.

16. The method of claim 13, wherein the herbal ointment is disposed within a transdermal patch.

17. The method of claim 13, wherein the herbal ointment is disposed within at least one of a roll-on stick and a spray applicator

18. The method of claim 13, wherein the herbal ointment is disposed within an electrode pad.
